(19) United States
(12) Patent Application Publication
(10) Pub. No.: US 2011/0124866 A1
(43) Pub. Date: May 26, 2011

(54) PROCESS FOR PREPARATION OF TADALAFIL

(75) Inventors: Lukasz Kaczmarek, Warszawa (PL); Pawel Pyc, Warszawa (PL); Katarzyna Badowska-Roslonek, Otwock (PL)

(73) Assignee: Zaklady Famaceutyczne Polpharma SA, Starogard Gdanski (PL)

(21) Appl. No.: 12/996,157

(22) PCT Filed: Jun. 3, 2009
(86) PCT No.: PCT/PL2009/000060

§ 371 Date: Feb. 2, 2011

(30) Foreign Application Priority Data
Jun. 3, 2008 (PL) P.385356

(51) Int. Cl. C07D 471/14

(52) U.S. Cl. 544/343

(57) ABSTRACT

Process for the preparation of tadalafil of high pharmaceutical purity is characterized in that the sequence of reactions comprising acetylation of methyl (1R,3R)-1-(1,3-benzodioxol-5-yl)-2,3,4,9-tetrahydro-1H-pyrido[3,4-b]indole-3-carboxylate with chloro acetyl chloride and cyclisation of the formed intermediate with methyl amine, is performed as a ‘one-pot’ process, without isolating the intermediate methyl (1R,3R)-1-(1,3-benzodioxol-5-yl)-2-(chloroacetyl)-2,3,4,9-tetrahydro-1H-pyrido[3,4-b]indole-3-carboxylate.

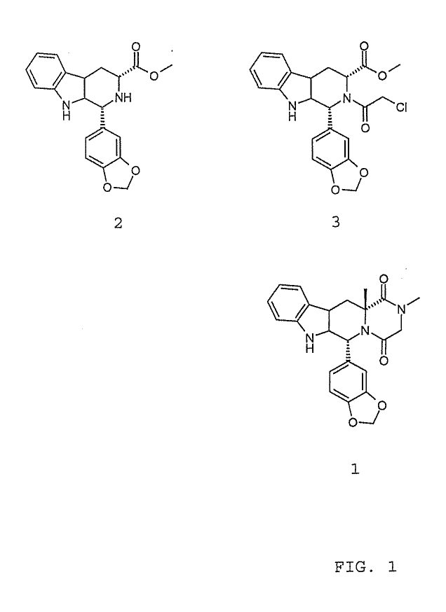

### CROSS-REFERENCE TO RELATED APPLICATIONS

The present Application is a US national phase of PCT/PL2009/000060 filed on Jun. 3, 2009 (“PCT Application”), which claims priority from Polish Application No. P. 385356 filed on Jun. 3, 2008, both of which are hereby incorporated by reference in their entirety into the present Application.

### BACKGROUND

The invention relates to the process for preparation of tadalafil starting from methyl (1R,3R)-1,2,3,4-tetrahydro-1-(3,4-dimethyldioxyphenyl)-9H-pyrido[3,4]indole-3-carboxylate or its hydrochloride.

Tadalafil, (6R-trans)-6-(1,3-benzodioxol-5-ilo)-2,3,6,7,12,12a-hexahydro-2-methyl-pyrazinol[1′,2′:1,6]pyrido[3,4-b]indole-1,4-dion (Formula 1), is a phosphodiesterase 5 inhibitor, marketed as CIALIS® in sexual dysfunction treatment.

The synthesis of tadalafil disclosed in EP 0740668 B1 comprises cyclisation of D-tryptophan methyl ester with piperonal in a presence of trifluoroacetic acid under Pictet-Spengler protocol, separation of cis and transisomeric mixture of methyl (1R,3R)-1-(1,3-benzodioxol-5-yl)-2,3,4,9-tetrahydro-1H-pyrido[3,4-b]indole-3-carboxylate (Formula 2) followed by acetylation of intermediate having the desired configuration cis with haloacetyl halide (e.g. chloroacetyl chloride) and subsequent cyclisation of obtained methyl (1R,3R)-1-(1,3-benzodioxol-5-yl)-2-(chloroacetyl)-2,3,4,9-tetrahydro-1H-pyrido[3,4-b]indole-3-carboxylate with methylamine (Formula 3).

Pictet-Spengler cyclisation between tryptophan methyl ester or its hydrochloride and piperonal in a presence of trifluoroacetic acid is described in many patents. This process may be carried out in different media, such as chlorinated hydrocarbons (EP 0740668 B1, WO 2005/068464), aromatic hydrocarbons (EP 0740668 B1, WO 2005/068464, WO 2006/110893) or in ester of lower carboxylic acids (WO 2006/110893).

Acetylation of methyl (1R,3R)-1-(1,3-benzodioxo1-5-yl)-2,3,4,9-tetrahydro-1H-pyrido[3,4-b]indole-3-carboxylate with acetyl chloride, usually is performed in a presence of tertiary amines or alkali metals carbonates or hydrocarbonates in chlorinated solvents, such as trichloromethane and dichloromethane using a haloacetyl halide (e.g., chloroacetyl chloride) (EP 0740668 B1) or ethers, for example tetrahydrofuran (e.g. using an amine, such as R₃NH_{2,} wherein R₃ is C₁-C₆ alkyl or hydro) (WO 2004/011463). The term “hydro” however does not seem to be defined in WO 2004/011463.

The alternate variety of solvents applied in hereinbefore described reaction comprises lower alcohols or acetone (WO 2004/011463), aromatic hydrocarbons, non-cyclic ethers and lower carboxylic acids esters (WO 2006/110893).

According to the disclosure of EP 0740668 B1, multi-step isolation of the product acetylated in anhydrous chloroform, consists of removing solvent by distillation and crystallization of the oily residue from ether.

In the Example of WO 2004/011463, acetylation of methyl (1R,3R)-1-(1,3-benzodioxol-5-yl)-2,3,4,9-tetrahydro-1H-pyrido[3,4-b]indole-3-carboxylate hydrochloride is performed in tetrahydrofuran-water solution until disappearance of starting material. The post-reaction mixture is condensed to reduce its initial volume to ca. 70%, the remaining solution is diluted with water and isopropyl alcohol, and again ca. 20% of solvents volume is removed. Form this mixture, methyl (1R,3R)-1-(1,3-benzodioxo1-5-yl)-2-(chloroacetyl)-2,3,4,9-tetrahydro-1H-pyrido[3,4-b]indole-3-carboxylate is isolated in a crystalline form.

The next synthetic step of the process for preparation of tadalafil, ie. cyclisation of methyl (1R,3R)-1-(1,3-benzodioxo1-5-yl)-2-(chloroacetyl)-2,3,4,9-tetrahydro-1H-pyrido[3,4-b]indole-3-carboxylate or its hydrochloride with methylamine according to EP 0740668 B1 may be accomplished suspending the former in alcoholic solution methanol/ethanol. After routine work-up procedure, which comprises, among other, removal of solvents under reduced pressure, the crude product is purified by flash chromatography followed by crystallization in methanol.

In WO 2004/011463 use of 40% aqueous methyl amine solution in cyclisation process, carried out in tetrahydrofuran, is disclosed. Analogically to EP 740668 B1, to remove the impurities the reaction mixture is subject to elaborate work-up procedure, including twofold addition of isopropanol and water mixture, followed by tetrahydrofuran distillation off. Obtained crystalline tadalafil is recrystallized from acetic acid to increase its purity.

The group of solvents used in cyclisation reaction until now, embraces, ethanol (WO 2005/068464, WO 2005/16030, WO 2007/052283), lower alkyl esters, nitriles and aromatic hydrocarbons (WO 2006/091975).

The limitations of hitherto published methods of tadalafil preparation mainly concern the necessity of intermediates isolation and solvents exchange in the following synthetic steps. The mentioned above drawbacks implicate additional chemical operations, increased energy input (distillation of solvents) and environmental pollution due to utilization of generated wastes. Use of various solvents in subsequent synthetic steps is the cause of additional efforts, undertaken to reduce the level of their residues in the final product, to be compliant with the purity requirements of the medicinal products authorities. In WO 2004/011463 use of the same solvent, tetrahydrofuran, in two following tadalafil synthetic steps, starting from methyl (1R,3R)-1-(1,3-benzodioxol-5-yl)-2-(chloro acetyl)-2,3,4,9-tetrahydro-1H-pyrido[3,4-b]indole-3-carboxylate or its hydrochloride is described. However, this method also requires performing laborious post-reaction mixture work-up procedure as well as isolation of the intermediate.

The pharmaceutical substances authorized for human use must meet the requirements established by International Conference on Harmonization of Technical Requirements for Registration of Pharmaceuticals for Human Use (ICH). These standards impose the necessity to develop new, more effective methods of tadalafil of high pharmaceutical purity synthesis, especially when compared to the processes known in the prior art. In ICH Topics [symbol] 3 C ‘Impurities: Residual Solvents’, acceptable daily dosages of residual solvents, PDE, are also set up, amount of which is less than 5000 ppm for the solvents of toxicity class 3.

Whenever hereafter a reference is made to ‘tadalafil of high pharmaceutical purity’, is to be understood substance including acceptable amounts of residual solvents, less than 0.1% of single impurity or less than 0.4% of total unidentified impurities.

### SUMMARY

Unexpectedly, the process has been found which overcomes discussed above inconveniences and provides tadalafil of high pharmaceutical purity. The process described above, illustrated in attached scheme, which starts with methyl (1R, 1R)-1,2,3,4-tetrahydro-1-(3,4-methylenedioxyphenyl)-9H-pyrido[3,4-b]indolo-3-carboxylate or its hydrochloride is to be carried out with the use of one solvent and without isolation of the intermediate.

The invention provides the process for preparation of tadalafil of high pharmaceutical purity characterized in that the sequence of reactions comprising acetylation of methyl (1R,3R)-1-(1,3-b enzodioxol-5-yl)-2,3,4,9-tetrahydro-1H-pyrido[3,4-b]indole-3-carboxylate with chloro acetyl chloride and cyclisation of the formed intermediate with methyl amine, is performed as a ‘one-pot’ process, without isolating the intermediate methyl (1R,3R)-1-(1,3-benzodioxol-5-yl)-2-(chloroacetyl)-2,3,4,9-tetrahydro-1H-pyrido[3,4-b]indole-3-carboxylate.

### DETAILED DESCRIPTION

In the preferred embodiment, the process is carried out in a solvent selected from a group comprising cyclic ethers and aliphatic ketones or the mixtures thereof.

The preferred cyclic ether is tetrahydrofuran and the preferred ketone is acetone.

According to ICH recommendations, acceptable PDE daily dosage is 5000 ppm for acetone (toxicity Class 3) and 720 ppm for tetrahydrofuran (toxicity Class 2).

The process for preparation of tadalafil according to the present invention is characterized in that to the suspension of methyl (1R,3R)-1-(1,3-benzodioxol-5-yl)-2,3,4,9-tetrahydro-1H-pyrido[3,4-b]indole-3-carboxylate (Formula 2) or its salt in a solvent, tertiary amine or alkali metal carbonate, preferably triethylamine is added, 1-10 molar equivalents, preferably 1,5-3 molar equivalents calculated to the starting compound of Formula 2. Chloro acetyl chloride is then added dropwise, while stirring at 0-80oC, preferably at reflux of the reaction medium, until substrate disappearance of Formula 2 is observed. Methylamine solution is subsequently added as 1-10 molar equivalents calculated to chloro acetyl chloride, preferably 8-10 molar equivalents, continuing stirring at 0-80° C., preferably at reflux of the reaction mixture. When the intermediate of Formula 3 is entirely consumed, solvent is distilled off and obtained product is recrystallized.

Elimination of the intermediate isolation and purification steps contribute to shortening of the process duration and obtaining tadalafil active substance of high pharmaceutical purity, which meets ICH standards. Chemical purity of the substance analyzed by high-performance liquid chromatography is more than 99.5% and even more than 99.9%. Residual solvents assay in the final product determined by gas chromatography is far less than 720 ppm, for the substance obtained in tetrahydrofuran and less than 5000 ppm, when the process was carried out in acetone.

The present invention is illustrated by the following examples, which should not be construed as any limitations of its scope.

### EXAMPLE 1

In 500 mL flask equipped with reflux condenser and magnetic stirrer, compound of Formula 2 was placed (7 g, 0.02 mol), then THF (350 mL) and triethylamine (4.2 mL, 0.03 mol) were added, the resulting mixture was heated to reflux. At reflux chloro acetyl chloride (2.2 mL, 0.028 mol) was added drop wise, stirring and heating were continued for 30 min. According to TLC analysis, the whole amount of substrate was consumed. To this reaction mixture 40% aqueous methylamine solution (7 mL, 5 eq.) was added at reflux, then stirring and heating were continued for 4.5 h. On TLC substrate signal was still present, therefore additional portion of methylamine solution (4.2 mL, 3 eq.) was added at reflux; this temperature was maintained for 3 h, until the substrate signal disappeared (TLC control). The solution was condensed under reduced pressure to dryness, resulting in 7.4 g of crude product, which was crystallized from acetone (300 mL). Tadalafil of 99.97% (HPLC) purity, in 4.1 g (53%) yield was obtained. THF residues were not detected.

### EXAMPLE 2

500 mL flask equipped with reflux condenser and magnetic stirrer, compound of Formula 2 as a salt with hydrochloric acid was placed (7.7 g, 0.02 mol), then THF (350 mL) and triethylamine (8.4 mL, 0.06 mol) were added, the resulting mixture was heated to reflux.

At reflux chloro acetyl chloride (2.2 mL, 0.028 mol) was added drop wise, stirring and heating were continued for 30 min. According to TLC analysis, the whole amount of substrate was consumed. To this reaction mixture 40% aqueous methylamine solution (7 mL, 5 eq.) was added at reflux, then stirring and heating were continued for 4.5 h. On TLC substrate signal was still present, therefore additional portion of methylamine solution (4.2 mL, 3 eq.) was added at reflux; this temperature was maintained for 3 h, until the substrate signal disappeared (TLC control). The solution was condensed under reduced pressure to dryness, crude product was crystallized from acetone (300 mL), yielding tadalafil of 99.95% (HPLC) purity and 3.9 g (50%) yield.

### EXAMPLE 3

500 mL flask equipped with reflux condenser and magnetic stirrer, compound of Formula 2 was placed (7 g, 0.02 mol), then THF (350 mL) and triethylamine (4.2 mL, 0.03 mol) were added, the resulting mixture was heated to reflux. At reflux chloro acetyl chloride (2.2 mL, 0.028 mol) was added drop wise, stirring and heating were continued for 30 min. According to TLC analysis, the whole amount of substrate was consumed. To this reaction mixture 2

M methylamine solution in THF (100 mL, 10 eq.) was added at reflux, then stirring and heating were continued for 10 h. The solution was condensed under reduced pressure to dryness, the residue was crystallized from acetone (300 mL), yielding tadalafil of 99.96% (HPLC) purity and 4.5 g (58%) yield. The contents of acetone (GC) was ca. 3300 ppm.

### EXAMPLE 4

500 mL flask equipped with reflux condenser and magnetic stirrer, compound of Formula 2 was placed (7 g, 0.02 mol), then acetone (350 mL) and triethylamine (4.2 mL, 0.03 mol) were added, the resulting mixture was heated to reflux. At reflux chloro acetyl chloride (2.2 mL, 0.028 mol) was added drop wise, stirring and heating were continued for 30 min. According to TLC analysis, the whole amount of substrate was consumed. To this reaction mixture 40% aqueous methylamine solution (11.2 mL, 8 eq.) was added at reflux, then stirring and heating were continued for 8 h. The solution was condensed under reduced pressure to dryness, resulting in 7.4 g of crude product, which was recrystallized from acetone. Tadalafil of 99.85% (HPLC) purity, in 2.6 g (33%) yield was obtained.

1. A process for the preparation of tadalafil of high pharmaceutical purity, wherein the sequence of reactions comprising acetylation of methyl (1R,3R)-1-(1,3-benzodioxol-5-yl)-2,3,4,9-tetrahydro-1H-pyrido[3,4-b]indole-3-carboxylate with chloro acetyl chloride and cyclisation of a formed intermediate with methyl amine, is performed as a ‘one-pot’ process, without isolating the formed intermediate methyl (1R,3R)-1-(1,3-benzodioxol-5-yl)-2-(chloroacetyl)-2,3,4,9-tetrahydro-1H-pyrido[3,4-b]indole-3-carboxylate, to obtain a crude product.

2. The process according to claim 1, wherein the process is carried out in a solvent selected from the group comprising cyclic ethers or aliphatic ketones or the mixtures thereof.

3. The process according to claim 2, wherein the solvent selected from the group of cyclic ethers is tetrahydrofuran.

4. The process according to claim 2, wherein the solvent selected from the group of aliphatic ketones is acetone.

5. The process according to claim 1 or 2, wherein the reaction is carried out at reflux.

6. The process according to claim 1 or 2, wherein the acetylation of methyl (1R,3R)-1-(1,3-benzodioxol-5-yl)-2,3,4,9-tetrahydro-1H-pyrido[3,4-b]indole-3-carboxylate with chloro acetyl chloride is performed in presence of a tertiary amine, used as a base, as 1-10 molar equivalents calculated relative to the methyl (1R,3R)-1-(1,3-benzodioxol-5-yl)-2,3,4,9-tetrahydro-1H-pyrido[3,4-b]indole-3-carboxylate.

7. The process according to claim 1 or 2, wherein methylamine is used in a proportion of 1-10 molar equivalents calculated with respect to chloro acetyl chloride.

8. The process according to claim 1 or 2, wherein the crude product is crystallized from acetone.

9. The process according to claim 6, wherein the tertiary amine used as a base, is triethylamine.

10. A process of manufacturing tadalafil comprising the steps as claimed in claims 6, 8 and 9.

11. The process according to claim 1 or 2, wherein the acetylation of methyl (1R,3R)-1-(1,3-benzodioxol-5-yl)-2,3,4,9-tetrahydro-1H-pyrido[3,4-b]indole-3-carboxylate with chloro acetyl chloride is performed in presence of a tertiary amine, used as a base, as 1,5-3 molar equivalents calculated relative to the methyl (1R,3R)-1-(1,3-benzodioxo1-5-yl)-2,3,4,9-tetrahydro-1H-pyrido[3,4-b] indole-3-carboxylate.

12. The process according to claim 1 or 2, wherein methylamine is used in a proportion of 8-10 molar equivalents calculated with respect to chloro acetyl chloride.

13. The process according to claim 1, wherein the crude product is crystallized.
